(19) United States
(12) Patent Application Publication
(10) Pub. No.: US 2004/0253284 A1
(43) Pub. Date: Dec. 16, 2004

(54) Sebum-adsorbent powder and use thereof

(76) Inventors: Masaakira Horino, Kanagawa (JP); Takeshi Arakawa, Saitama (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(21) Appl. No.: 10/822,870

(22) Filed: Apr. 13, 2004

(30) Foreign Application Priority Data
Apr. 18, 2003 (JP) 2003-114156

(51) Int. Cl. A61K 7/00

(52) U.S. Cl. 424/401

(57) ABSTRACT

There is provided a powder having a base substance usable as a powder for cosmetics, a layer of hydroxyapatite which exists directly on the surface of the base substance and a layer of zinc oxide which exists directly on the surface of the layer of hydroxyapatite, and containing 5 to 30% by weight of the hydroxyapatite and 10 to 50% by weight of the zinc oxide based on the total weight of the powder. This powder is suitable for use in cosmetics. As the powder has an improved properties for cosmetics, or adsorbent property of unsaturated fatty acids or sebum, it is also useful for a sebum-adsorbent agent, a body deodorant or the like as well as the cosmetics.

### BACKGROUND OF THE INVENTION

The present invention relates to a novel powder having a particularly excellent sebum-adsorbent property, and to cosmetics, a sebum-adsorbent agent, a body deodorant and the like, each employing the novel powder. More particularly, it relates to a composite powder comprising a base substance usable as a powder for cosmetics, a hydroxyapatite coating the surface of the base substance and a zinc oxide coating the surface thereof, and particularly suitable for use in cosmetics, such as a sebum-adsorbent agent, a body deodorant, or the like. It is possible to provide cosmetics, a sebum-adsorbent agent, a body deodorant and the like by the use of the powder in the present invention.

### DESCRIPTION OF THE RELATED ART

The sebum secreted from the skin affords an emollient effect to the stratum corneum of the skin which keratinizes normally, prevents intrusion of toxic substance or bacteria from the outside, and controls the release of substances, such as water, out of the body. However, excess secretion of the sebum has a demerit that it may be a factor causing the makeup to come off which leads to some phenomena, for example, a “shiny” or “drab” appearance of the skin, or a “unevenness”, “rumpling”, or “disappearance” of the makeup itself, and the like caused by chronological changes in the cosmetic film that is coated on the skin, and it may be also due to the formation of peroxides by the oxygen contained in atmospheric air. Similar to photo-degradation of fats and oils, such formation of peroxides, if intruded into the skin, operates as irritants that may cause inflammation or a keratinization disorder, blemishes, or freckles on the skin. So, there is raised a demand for developing a technology for effectively removing sebum, wherein it takes account of safeness of the skin so as not to impose an excessive burden on the skin.

A variety of investigations have already been conducted from the viewpoint of improving long wear for makeup. For example, should highly moisture-absorbent or oil-absorbent substances, such as porous silica, calcium carbonate, magnesium carbonate, and crystalline cellulose, be mixed into cosmetics, moisture and sebum components on the skin become adsorbed, which leads to a shortage of skin emollient components and causes a dry skin feeling, and a tightness of skin or itchiness of skin. This phenomenon is most likely to occur with persons with dry skin and normal skin, in particular with persons living in an environment and the like (for example, persons working in an office) where perspiration (sweat) or sebum are not apt to be secreted. When used on oily skin, these substances have demerits, which are apt to present luster due to excess sebum, thus giving rise to a “shiny” look notedly in the finished makeup. On the other hand, when used on dry skin and normal skin, in case of using oily products, such as oily foundation, these substances have demerits as same as above, which are apt to present luster by the progress of the wet phenomenon due to oily components (oily agents) contained in the oily products, thus giving rise to a “shiny” look in the finished makeup.

Although cosmetics admixed with fluorine-processed powders (powders treated with fluorine) have been proposed for improving the long wear for makeup, cosmetics making use of these powders, while not being wetted with sebum or perspiration, exhibits strong water- or oil-repellency thus adhering to the skin in a small amount and causing the cosmetic film to be “rumpled” due to slipping of the powder on the skin thus impairing the makeup effect.

The cosmetics employing the powders treated with silicone are high in water repellency and also exhibit resistance against perspiration and moisture. However, the silicone oil, and the silicone oil or its derivative which is applied for surface treatment of the powder are low in oil resistance, basically due to the basic structure of the silicone oil. Consequently, in the case of a person with an oily skin or oily dry skin, the makeup may come off from the entire face due to excess secretion of sebum. On the other hand, in the case of a person with normal skin, the makeup may come off at the T- or V-zone of the face, so that it is difficult to prevent the makeup from coming off due to secretion of sebum.

A film forming polymer material may be utilized for improving long wear for makeup. As representative of this type of substance, an acryl-silicone based graft polymer has been proposed. This is produced and obtained by radical polymerization of a dimethyl polysiloxane composite which has a radical polymerizability at one terminal end of the molecular chain with a radical polymerization monomer composed essentially of an acrylate or a methacrylate, and forms a cosmetic film superior in water- and oil-repellency so that it is made practicable in a non-aqueous foundation. However, the cosmetics employing these polymers, from the viewpoint of enveloping the skin, leave many problems of a physiological aspect in the skin when taking into consideration the everyday biological activity on the surface of the skin. Moreover, in powder products in which the skin-forming capability cannot be used effectively, long wear is difficult to be improved.

There are also proposed a zinc oxide coated substance in which a base substance is coated with an amorphous zinc oxide, and there are reported a powder exhibiting good spreadability without detracting from the fatty acid-solidifying capability and an external agent for skin, using this powder (see Japanese Patent Kokai Publication JP-A-9-227792). However, the amount of adsorption of an oleic acid, which the zinc oxide coated substance exhibits, is approximately the same as that of the normal porous silica and is not particularly excellent in adsorption of the free fatty acids. Moreover, the amount of adsorption of an artificial sebum, which this coated substance exhibits, is lesser than the porous silica beads, and furthermore this coated substance takes approximately 30 minutes to solidify fatty acids, thus presenting difficulties in coping with the oily skin or ultra-oily skin.

On the other hand, there are reports of a composite, which is inclusive of one or more oxides and/or their hydroxides in the inter-layer of the clay mineral or an inter-layer metal inclusive composite adsorbing selectively only free fatty acids (see Japanese Patent Kokai Publication JP-A-10-87420). This material uses a water-swollen type of clay mineral and is produced by the reaction in the sol-state. As may be understood from the description of the Examples thereof, the reaction takes place in the diluted solution. Therefore, this method is costly in production per batch and is economically unmeritorious. Moreover, since the reaction occurs in the sol-state, the washing process is extremely time-consuming, when a regular filtering, washing, and drying process is used. The process is not only extremely time-consuming, but also the obtained product aggregates strongly, thereby not obtaining the result that is expected. In addition, a freeze-drying process is essential so that production becomes very expensive as another weak point. Although AL pillars are formed in the inter-layer of the clay mineral, the variations of content of intercalated oxide or hydroxide generates different states of pillars because of differences between lots of the clay mineral, it is usually difficult to obtain products in the same stable quality.

Under these circumstances, the present applicant has been proposed a composite powder in which a base substance has a hydroxyapatite layer on the surface of particle of the base substance and further has a zinc oxide layer on the surface thereof for solving the above problem (see Japanese Patent Kokai Publication JP-A-2002-20218). The powder is reported to be superior in sebum-adsorbent property, and has an antibacterial effect and a property for adsorbing body odor components. Although the powder is now used for the cosmetics having sebum-adsorbent property and the like, further improvements in properties for cosmetics (effects of cosmetics), such as improvement of long wear for makeup, suppression of “shiny” look and improvement of aesthetic feel, or an adsorbent property of unsaturated fatty acid or sebum are desired.

### SUMMARY OF THE INVENTION

It is an object of the present invention to provide a powder obtained by improving the composite powder described in Japanese Patent Kokai Publication JP-A-2002-20218, that is, a powder improved in properties for cosmetics (effects of cosmetics) and an adsorptive property of unsaturated fatty acid or sebum.

It is an another object of the present invention to provide cosmetics comprising the powder described above.

The present inventors have conducted perseverant researches and studies to the powder described in Japanese Patent Kokai Publication JP-A-2002-20218 towards solving the above problem in detail, and found that properties for cosmetics (effects of cosmetics), an adsorbent property of free fatty acid (in particular the unsaturated fatty acid) or sebum, and solidifying capability to those fatty acids can be improved by increasing the usage rate (content) of zinc oxide widely against the prior product, particularly using (containing) it in the range of 10 to 50% (by weight), and the above problem can be solved. The above variety of knowledges have led to the completion of the present invention.

According to an aspect of the present invention, there is provided a powder having a base substance usable as a powder for cosmetics, a layer of hydroxyapatite which exists directly on the surface of said base substance and a layer of zinc oxide which exists directly on the surface of said hydroxyapatite, and containing 5 to 30% (by weight) of the hydroxyapatite and 10 to 50% (by weight) of the zinc oxide based on the total weight of said powder. Moreover, the powder is suitable as a powder for cosmetics, that is, the powder can be applied to the cosmetics by admixing, or to other fields of use. For example, since the powder has the property of adsorbing sebum components and the effect of deodorizing the body odor, the powder can be used for a sebum-adsorbent agent, a body deodorant and the like, therefore, the present invention contains these embodiments.

Now a sebum-adsorbent agent means an adsorbent agent for sebum, or an agent for adsorbing sebum.

Meanwhile, the powder of the present invention includes, but is not limited to the aforementioned three components (that is the base substance, the hydroxyapatite and the zinc oxide) at the above composition ratio, and the powder of the present invention may compose other components or other structures, as far as the effect in the present invention is obtained or the object of the present invention is not obstructed. As a matter of course, these contents are contained in the powder of the present invention.

Meanwhile, as stated above, the sebum-adsorbent agent means a substance used for adsorbing, solidifying, or congealing the sebum of an animal, especially a human being. On the other hand, the body deodorant means a substance used for absorbing, solidifying, fixing, or deodorizing at least one of the components of disagreeable odor emitted through the skin of an animal, especially the skin of a human being, or from the epidermic cell (for example, due to perspiration (sweat) or effect of microorganisms and the like). In particular, the body deodorant may be used as the powder (is) mixed into the skin cosmetics or used as the powder mixed for deodorizing body odor separate from cosmetics.

In the present invention, the body odor (smell) components compose of a broad sense of body odor components which is emitted from the animal, especially the human body (see Seiichi Izaki, What is Body odor—its Cause and Prevention, Fragrance Journal, 1990-7, p.22 to 26 (1990); Yuuichi Yamamura, Body odor, ‘Modern Dermatology 2B’ Whole Body and Skin 2, edited by Yuuichi Yamamura, Jun Kukita, Eishun Sano, Makoto Seiji, published by NAKAYAMA SHOTEN, Tokyo, 1981, 163).

### PREFERRED EMBODIMENTS OF THE INVENTION

In the following, a preferred embodiment of the present invention is explained in detail.

The present invention comprises a powder (composite powder) having a base substance usable as a powder for cosmetics, a layer of hydroxyapatite which exists directly on the surface of said base substance and a layer of zinc oxide which exists directly on the surface of said layer of hydroxyapatite, and containing 5 to 30% (by weight) of the hydroxyapatite and 10 to 50% (by weight) of the zinc oxide based on the total weight of said powder, that is, the powder of the present invention or the like. The following explanation is centered about the powder of the present invention. The powder of the present invention is not limited thereto.

(Powder of the Present Invention)

The powder (composite powder) of the present invention has a basic structure which is comprised of a layer of hydroxyapatite (hydroxyapatite layer) which exists directly on the surface of the base substance described above and a layer of zinc oxide (zinc oxide layer) which exists directly on the surface of this layer of hydroxyapatite (hydroxyapatite layer) at the specific composition ratio. More preferably, low crystalline zinc oxide, amorphous zinc oxide or a mixture thereof is used as zinc oxide. In case of forming the powder (composite powder) by coating this zinc oxide on the above hydroxyapatite layer, the powder highly useful as cosmetics can be formed. The base substance can be employed for a powder usable as cosmetics, both inorganic and organic, and may be used in the form of variety of composite powders, such as inorganic-inorganic powders, organic-organic powders, inorganic-organic powders, and the like. Among inorganic powders, there are clay minerals, metal oxides, metal hydroxides and composite materials comprising these materials, and the composite material(s) of one or more these inorganic material(s) with one or more organic material(s). As the organic powders, those usable for the base substance of variety of cosmetics may be used. As a matter of course, natural products or synthetic clay minerals may be contained in the clay minerals. It is also possible to use a composite material of the organic and inorganic powders, that is, the organic-inorganic composite powders. In the case of using them in the cosmetics and the like, one or more of these powders may, of course, be used.

There is no particular limitation to the particle shape. For example, there may be variety of shapes, such as lamellar shape, scale-like shape, plate-like shape, spherical shape, spindle shape, X-shape, astral shape, petal shape, starfish-like shape, ribbon-like shape, needle-like shape, hemispherical shape, bar-like shape and the like. The lamellar, scale-like, plate-like or bar-like shape is particularly preferred in that it is ready to produce the same light reflection curve as that obtained on the surface of skin.

The size of the particle in the powder used for the base substance, expressed as mean particle size, is preferably on the order of 0.1 to 600 μm, more preferably on the order of 0.3 to 140 μm, moreover preferably on the order of 1 to 80 μm and most preferably on the order of 2 to 50 μm.

In the present invention, the clay minerals used in the base substance, may be inclusive of synthetic products, and may be enumerated by kaolins, such as kaolionite, dekkite, nacrite, halloysite, antigorite, chrysotile and the like, smectites, such as pyrophyllite, montmorillonite, nontronite, sabonite, hectorite, bentonite and the like, illites, such as sericite, white mica, black mica, lithia mica, gold mica, synthetic mica, synthetic sericite and the like, silicates, such as calcium silicate, magnesium silicate, magnesium aluminum silicate and the like, magnesium silicates, such as talc, serpentine and the like, natural and synthetic zeolite, tourmaline and the like. If metal oxides are used as the base substance, single component powder, such as silica, alumina, titanium oxide, cerium oxide and the like, bismuth oxychloride, barium sulfate and the like, may be used. In particular, plate-like shaped or scale-like (scalar) shaped products are desirable. Moreover, the form of composite material may be used. The composite oxides may be enumerated by multi-layered composite material, such as silica-titanium dioxide, silica-barium sulfate, silica-zinc oxide, silica-titanium dioxide-silica, silica-cerium oxide-silica, silica-zinc oxide-silica and the like, composite powders, such as titanium mica, titanium dioxide-silica, titanium dioxide-glass flake, colored inorganic pigment-titanium dioxide-mica, organic pigment-titanium mica; irised foil pearl pigment, titanium dioxide-barium sulfate, titanium dioxide-talc, zinc oxide-mica, zinc oxide-talc, bismuth oxychloride-mica, titanium dioxide-cerium oxide-alumina-talc, titanium dioxide-zirconium oxide-alumina-silica, titanium dioxide-alumina-silica-talc and the surface of these composite powders treated with aluminum hydroxide, aluminum oxide, magnesium oxide, magnesium hydroxide, calcium hydroxide, calcium oxide, silica, barium sulfate, and hard capsules, such as titanium dioxide encapsulating PMMA, zinc oxide encapsulating PMMA, cerium oxide encapsulating PMMA, dye encapsulating polystyrene, color pigment encapsulating PMMA and the like.

Among the organic powder used as the base substance, there are variety of powders usable for cosmetics, such as nylon powders, polyethylene powders, polypropylene powders, polystyrene powders, vinyl acetate powders, polymethacrylic acid ester powders, polyacrylonitrile powders, silicone rubber powders, silicone resin powders, silicone elastomer powders, cellulose powders and the like. The organic-inorganic composite powders may be enumerated by polyethylene-zinc oxide, polyethylene-titanium dioxide, polyethylene-aluminum hydroxide, polyethylene-aluminum hydroxide-PMMA and the like. If the organic-organic composite powders are used, nylon-cellulose may be used.

There is no particular limitation to the hydroxyapatite used in the present invention. It is defined as calcium phosphate, with Ca/P=0.5 to 2.0 (in molar ratio), having an apatite structure (see Fragrance Journal, p144 to p148, 1999 January). Such calcium phosphate may be used in the present invention.

The hydroxyapatite, that is coated on the surface of base substance, has the action of specifically adsorbing free fatty acids, in particular unsaturated fatty acids. It may be presumed that this free fatty acid operates as a factor responsible for promoting makeup to come off due to the action of specifically lowering the melting point of the sebum. The hydroxyapatite does not exhibits the sebum-solidifying capability, but it adsorbs free fatty acid secreted from the skin and prevents the makeup from coming off due to the lowering of the melting point of the sebum. The hydroxyapatite also plays the role of keeping the skin clean by the adsorptive action of peroxides generated due to oxidation of sebum secreted from the skin.

The hydroxyapatite coating that is coated on the surface of the base substance increases its crystallinity by heat treatment, with the crystallinity being higher, a higher heat treatment temperature being necessary. Particularly, in case that the heat treatment temperature is 1000° C., the crystallinity of hydroxyapatite is remarkably high and the pore size of hydroxyapatite enlarges to approximately 5 times of that, and also the amount of pore of hydroxyapatite tends to decrease. However, since the amount of the free fatty acid adsorbed is inversely correlated with the heat treatment temperature, it is more desirable not to apply heat treatment.

Although there is no limitation to the hydroxyapatite used in the present invention, as described above, Ca₅(PO₄)₃(OH), Ca₁₀(PO₄)₆(OH)₂, Ca₄(P₄)₂O, Ca₁₀(PO₄)₆F₂, Ca₃(PO₄)₂ and the like are cited preferable in view of safety.

As for the amount of hydroxyapatite used for coating, preferably on the order of 5 to 30 wt %, more preferably on the order of 5 to 20 wt % and most preferably on the order of 8 to 15 wt % are selected based on the weight of total composition of the powder in the present invention, in particular, the total weight of the base substance, the hydroxyapatite and the zinc oxide. The coating amount less than 5% is insufficient to keep the skin clean due to the excessively small amount of the adsorption. If the coating amount exceeds 30 wt %, the amount of adsorption cannot be expected to be increased in proportion to the amount used for coating. Moreover, the slipperiness (lubricant property) on the skin is unchanging. Although there is no limitation to the thickness of the coating layer of hydroxyapatite, preferably on the order of 0.05 to 10 μm and more preferably on the order of 0.1 to 5 μm are selected. Meanwhile, the layer can be formed by coating the surface of the base substance particle with (many) hydroxyapatite particle. In this case, the particle shape of hydroxyapatite is preferably spicular. In such case, for the length thereof on the average, preferably 0.1 to 10 μm or so, more preferably 0.1 to 5 μm or so and most preferably 0.1 to 2 μm or so are selected, and for the breadth thereof on the average, preferably 0.01 to 0.06 μm or so, more preferably 0.01 to 0.04 μm or so and most preferably 0.02 to 0.04 μm or so are selected. Further, as for the hydroxyapatite, a porous hydroxyapatite in which the pore size of surface of particle, expressed as mean value, is 1 to 5 nm or so can be selected.

After coating the surface of the base substance with a hydroxyapatite, a zinc oxide can be coated on the surface of the hydroxyapatite coating layer. As a zinc oxide for coating, since zinc oxide having high crystallinity is low in both free fatty acid-adsorbent properties and sebum solidifying capability for solidifying the sebum, it is desirable to use low crystalline zinc oxide, amorphous zinc oxide or mixtures thereof. Meanwhile, in the present invention, as for the layer of zinc oxide (zinc oxide layer) formed on the surface of the hydroxyapatite layer, the layer can be formed by coating the surface of the layer formed by the hydroxyapatite particle with (many) zinc oxide(s) particle. In this case, the particle size, expressed as mean particle size, is preferably on the order of 0.001 to 1 μm, more preferably on the order of 0.01 to 0.05 μm, and most preferably on the order of 0.01 to 0.03 μm. Moreover, as for the specific surface area of the zinc oxide, preferably 105 to 500 m² /g or so, more preferably 120 to 300 m² /g or so and most preferably 140 to 180 m² /g or so are selected. Further, in the zinc oxide that is subjected to firing process or heat treatment process, particle growth occurs, its surface adsorption site decreases, its crystallinity increases, or decrease of amount of adsorption occurs. Therefore, it is preferabe to produce by a method for producing zinc oxide in aqueous phase without heat treatment process.

Meanwhile, “low crystalline”, in the low crystalline zinc oxide, means the state in which crystalline planes are not arrayed neatly in numerous random orientations of small-sized crystals. The X-ray diffraction peak width is determined by the variable orientations (arraying states) of individual micro-crystallites and the crystalline size. In the case of the large crystalline size, light scattering is small, whereas, in the case of the small crystalline size, the peak width is broad. The crystalline size may be obtained from Scherrer's equation. Although this equation is lowered in reliability when the numerical value exceeds 1000 Å (100 nm), it has been seen that the crystalline size of the low crystalline zinc oxide is evidently small in crystalline size as compared to that of the crystalline zinc oxide.

The “amorphous”, in the amorphous zinc oxide, is defined as being in the solid state in which the crystal lattice (periodic array of atoms) is hardly noticed or as being a solid which fails to give an X-ray diffraction image even if there exists certain periodic array of atoms.

Although the sebum composition differs with the sex and with age, with the result done by the researches of SHISEIDO KK, in the case of a female, the sebum comprise free fatty acid on the order of 7 to 13%, squalene on the order of 11 to 17%, wax on the order of 14 to 17%, triglyceride on the order of 47 to 55%, diglyceride on the order of 3 to 5%, monoglyceride on the order of 0.7 to 1.2%, stearin esters on the order of 1.4 to 1.5% and free stearins on the order of 1.4 to 1.5%. Therefore, if approximately 7 to 13% of free fatty acids in the sebum composition only is adsorbed, many other sebum components remains on the skin, such that the prevention of the makeup from coming off is done insufficiently, and hence it is necessary to adsorb, solidify or congeal other secreted sebum components to prevent the sebum from being fluidized.

The powder of the present invention, as compared to the prior products (the powder of Japanese Patent Kokai Publication JP-A-2002-20218 described above or the like), has particularly superior features in specifically adsorbing free fatty acid in the sebum composition at a larger amount and in preventing the melting point of the skin from being lowered, and also in exhibiting higher sebum-solidifying capability with higher capability of adsorbing fatty acid (fatty acid-adsorbing capability), through the interaction between the action of adsorbing the other sebum components, such as diglyceride or triglyceride and ester, and the sebum-solidifying capability. Moreover, the powder of the present invention can keep the skin clean because it has properties of forming a strong cosmetic film that prevents the makeup from coming off and adsorbing the peroxides generated by oxidation of sebum, and having antibacterial activities. Further, in case of using the powder of the present invention for a cosmetic, the cosmetic film obtained (formed) is superior in transparency, exhibits the same pattern as that of the skin's spectroscopic curve, prohibits non-transparency on the makeup finish or the whitish powder floating on the makeup finish, and has a soft focus effect. Moreover, in so far as the cosmetic effect is concerned, its adherency to the skin are given and uniform skin feeling are also given since complexion becomes blight, while the aesthetic feel is also equivalent to that of the scale-like powders routinely used in the cosmetics. In particular, properties for cosmetic (effects of cosmetic) whose improvement is expected in the prior products, such as improvement of long wear for makeup, suppression of “shiny” look and improvement of aesthetic feel, can be improved.

The amount of zinc oxide used in the powder of the present invention is preferably on the order of (approximately) 10 to 50 wt %, more preferably on the order of (approximately) 15 to 30 wt % and most preferably on the order of (approximately) 15 to 20 wt % based on the total composition (weight) of the powder, in particular the total weight of the base substance, the hydroxyapatite and the zinc oxide involved. In case of the amount of zinc oxide less than 10 wt %, it takes not less than approximately 2 minutes to solidify, and further, since soft gell structure is formed by asdorption of unsaturated fatty acid or sebum on the surface of composite powder, sufficient long wear for makeup is hard to obtain. The amount of zinc oxide exceeding 50 wt % is undesirable in that it takes not less than approximately 2 minutes to solidifying sebum, that is, the sebum-solidifying capability tends to be lowered due to interaction, and the effect of filler is hard to produce, though the aesthetic feel at the time of application does not change.

In the powder of the present invention, with regard to the composition ratio (weight), in particular, the composition ratio among the base substance, the hydroxyapatite, and the zinc oxide, preferably 20 to 85:5 to 30:10 to 50, more preferably 50 to 80:5 to 20:15 to 30 and most preferably 65 to 77:8 to 15:15 to 20.

In the powder of the present invention, the surface thereof can be treated. The surface thereof may be treated by a method of surface treatment, such as fluorine treatment, silicone treatment, metallic soap treatment, lecithin treatment, collagen treatment, ester treatment, chitosan treatment, lauroyl lysine treatment, copolymer of acrylic•silicone treatment, trimethylsiloxy silicate treatment, agar treatment and fluoro•modified silicone treatment. The method of surface treatment is not necessarily limited thereto. Moreover, one or more of these method of surface treatments may be selected. Particularly, in the present invention, as for the agent of the surface treatment, the surface treatment which is hard to cause a decrease of amount of adsorption is preferably selected, and based on the formability of the powder of the present invention which is superior to that of the prior powder, silicone treatment or fluorine treatment is preferably selected in view of the adjustment of filling property of powder, or the like.

The powder of the present invention can be produced without particular difficulties by, for example, the following method:

A liquid comprising a base substance dispersed therein is added to calcium acetate and is heated to 85° C. and admixed with a mixed solution of sodium hydroxide and disodium hydrogenphosphate (Na₂HPO₄). The pH value then is adjusted to approximately 9 to 10. The resultant product then is added to a sodium hydroxide solution and the pH value is adjusted to approximately 11 to 12. The resultant product is kept at approximately 85° C. and cured. After the end of curing, the solution is cooled and set at a temperature of approximately 60 ° C. When the temperature is at 60° C., the reaction mixture is adjusted to the pH value of approximately 12 by adding a 5N sodium hydroxide solution thereto. As the pH value is adjusted to and kept at approximately 12, a 1M zinc chloride solution and a 5N sodium hydroxide solution are dripped into the reaction mixture simultaneously. After that, the reaction mixture is cooled, filtered and washed with water repeatedly. The reaction product is dried at 120° C. for 16 hours or so and pulverized to obtain a powder of the present invention. The powder of the present invention, obtained as described above, specifically adsorbs the free fatty acids while simultaneously adsorbing and solidifying other sebum components, while maintaining its aesthetic feel, and is superior in the long wear effect for makeup, skin cleanliness (cleanness of the skin) and skin feeling, and in antibacterial properties.

Thus, the coated substance of the present invention is suited as a starting material for preparations of cosmetics and drugs. In particular, it is useful as a starting material for cosmetics since it is excellent in long wear for makeup and in antibacterial properties, while maintaining skin cleanliness and skin feeling. Moreover, it may be used as a sebum-adsorbent agent or as a body deodorant.

(Cosmetics)

The cosmetics of the present invention have features in that it comprises the powder of the present invention as described above. The cosmetics of the present invention can be applied to any agent form known for ordinary cosmetics, in particular it is not limited. These cosmetics may be applied to, for example, basic cosmetics, such as cream, emulsion, lotion, cleansing gel, sun-cam lotion and the like, solid water-in-oil cosmetic, solid oil-in-water cosmetic, base makeup agents, such as under-makeup, emulsion for T-zone, water-in-oil, oil-in-water and the like of cream or emulsion, foundation, such as emulsified foundation (emulsion or cream type), powder foundation, gel foundation, and the like, point makeup agents, such as eye-shadow, water-in-oil cilia cosmetic (such as mascara), lip cream, rouge brusher, concealor,lip gloss, lip color, nail color and the like, powder products, such as talcum powder, caramine lotion, baby powder, body powder, deodorant powder, antiperspirant powder, fragrance powder, face powder and the like, pack products, such as peel off pack, muddy pack, gel pack and the like, hair treatment products, such as tonic, shampoo, rinse and the like, soap and bath agent. The cosmetics of the present invention can also be applied to wet tissues, oil removing sheets or makeup removing agents and the like. In particular, the cosmetics of the present invention can be more desirably enumerated by makeup cosmetics, basic cosmetics and sweat controlling cosmetics (especially deodorant cosmetics and the like).

The reason is that the powder of the present invention exhibits a deodorant effect and antibacterial properties and, when the coated substance of the present invention is used as cosmetics, it exhibits a high adsorptive power for free fatty acids and sebum components, while being superior in long wear for makeup and skin feeling.

When the powder of the present invention is composed in cosmetics, there is no particular limitation to the amount of mixing in the cosmetics since it may be suitably selected depending on the type of the cosmetics. In general, preferably on the order of 0.01 to 90 wt %, more preferably on the order of 0.05 to 50 wt % and most preferably on the order of 0.1 to 30 wt % may be mixed in the entire cosmetics.

In addition to the powder of the present invention, those components used in routine cosmetics may be used. These components may be enumerated by hydrocarbons, such as petrolatum (vaseline), micro-crystalline wax, ceresin, squalane, fluid paraffin and the like, higher alcohol, such as cetanol, stearyl alcohol, olein alcohol and the like, fatty acids, such as stearic acid, palmitic acid, behenic acid and the like, triglycerides, such as beef tallow, olive oil and the like, esters such as myristic acid octyl dodecyl, dimethyl octanoic acid hexyldecyl, myristic acid isopropyl and the like, polyhydric alcohols, such as glycerine, 1, 3-butylene glycol and the like, surfactants, such as nonionic surfactants, anionic surfactants, amphoteric surfactants, cationic surfactants and the like, ethanol, thickeners, such as carboxy vinyl polymer, carboxy methyl cellulose sodium and the like, antiseptics, UV light absorbents, antioxidants, dyes and powders.

(Sebum-Adsorbent Agent)

The present invention contains a sebum-adsorbent agent. In particular, the present invention may be applied with the intention or the power of adsorbing, solidifying or congealing the sebum components of a human being. Therefore, the sebum-adsorbent agent also encompasses the aforementioned cosmetics. The product of the present invention may also be used as a sweat controlling agent in addition to the cosmetics. The content of the powder to be mixed to the sebum-adsorbent agent can be selected suitably. If the product of present invention is used for objects other than cosmetics, the amount of mixing used in the cosmetics can be used as reference values.

(Body Deodorant)

Usually, the body deodorant can be mixed into cosmetics for adsorbing, solidifying or congealing the body odor (smell) and the like, that is undesirable odor components emitted by perspiration or microorganisms through the skin of animals, in particular the skin of human being, and can decrease the odor. According to the present invention, a body deodorant is a substance (agent) used for adsorbing or solidifying at least one odor component emitted in particular from the human body through the skin, and the like, for reducing the odor (smell). The use thereof to the cosmetics is as described above. It can be affected by using the formulation customarily used or known as deodorant or antibromic agent (see Yoshihiro Ohhata, Tendency and Task of Recent Body Deodorant Products, Fragrance Journal, 1990-7, p-61 to 69, 1990). The amount of mixing of the powder of the present invention can be selected suitably depending on the species of agent form and the like. Usually, the amount of mixing, which is shown in the above-described cosmetics is referred.

This application is based on the Japanese Patent Application Serial No.2003-114156, field on Apr. 18, 2003, which is incorporated herein by reference in its entirety.

### EXAMPLES

The present invention is explained in detail by referring to the following Examples, Comparative Examples and Contrastive Examples.

### Example 1

### Preparation of Powder-1

152 g of sericite (mean particle size: 8 μm, plate crystal) were dispersed in 2000 ml of purified water in the reaction vessel. To the resultant liquid dispersion 51.84 g of calcium acetate were added and the resultant mixture was heated to 85° C. When the temperature is 85° C., a solution obtained on dissolving 9.6 g of sodium hydroxide and 25.7 g of disodium hydrogenphosphate in 320 ml of purified water was added to the heated mixture to adjust the pH value of the solution to 9.4. A solution obtained on dissolving 2.26 g of sodium hydroxide in 226 ml of purified water was then added to the mixture to adjust the pH value thereof to 11.4. The reaction mixture was subjected to reaction and curing for one hour. When the curing was finished, the reaction mixture was cooled to 60° C., and 560.7 ml of 1M zinc chloride was added drop wise thereto, as the pH value was kept at 12, using 224.3 ml of 5N sodium hydroxide solution. At the end of the dripping, the reaction mixture was cooled, filtered and washed with water repeatedly. The reaction product was dried at 120° C. for 16 hours and pulverized to obtain a coated product (powder) of the present invention.

### Examples 2 to 4 and Comparative Example 1

### Preparation of Powder-2 to 5

The powder of the present invention was prepared by the way shown in Example 1 except using each components at the usage described in the section of Example 2 in the following Table 1 in place of the usage of each component in the above Example 1 (Example 2). Next, the various powders were prepared in the same way as described above, based on the usage of each components which were described in the section of Example 3 or 4 or Comparative Example 1 in the following Table 1 (Examples 3 and 4 and Comparative Example 1).

Meanwhile, the percentage of zinc oxide content (% by weight) can be determined by X-ray fluorescence analysis or the like.

TABLE 1
Mixing amount (usage) of each components in production of various powders

| | | Example 1 | Example 2 | Example 3 | Example 4 | Comparative |
|---|---|---|---|---|---|---|
| | | (containing | (containing | (containing | (containing | Example 1 |
| | | 20%* of | 15%* of | 10%* of | 30%* of | (containing |
| components | | zinc oxide) | zinc oxide) | zinc oxide) | zinc oxide) | 5%* of zinc oxide) |
| purified water (at the time of dispersing | | 2000 | 2000 | 2000 | 2000 | 2000 |
| sericite) (ml) | | | | | | |
| sericite (mean particle size: 8 μm, plate | | 152 | 171 | 190 | 121.6 | 190 |
| crystal) (g) | | | | | | |
| calcium acetate (g) | | 51.84 | 58.32 | 64.8 | 41.5 | 64.8 |
| sodium | purified water (ml) | 320 | 360 | 400 | 284.2 | 400 |
| hydroxide-disodium | sodium hydroxide (g) | 9.6 | 10.8 | 12 | 8.5 | 12 |
| hydrogen phosphate | disodium | 25.7 | 28.9 | 32.12 | 22.8 | 32.12 |
| aqueous solution | hydrogenphosphate (g) | | | | | |
| sodium hydroxide | purified water (ml) | 226 | 243 | 270 | 187.6 | 270 |
| aqueous solution | sodium hydroxide (g) | 2.26 | 2.43 | 2.7 | 1.89 | 2.7 |

| | | | | | |
|---|---|---|---|---|---|
| 5 N sodium hydroxide solution (ml) | 224.3 | 177.6 | 124.3 | 404.6 | 59.2 |
| 1 M zinc chloride solution (ml) | 560.7 | 444.0 | 310.8 | 1011.5 | 148.0 |

*wt %

### Example 5

### Various Evaluation Tests for Powder

(Test Method)

A. Amounts of Adsorption of Oleic Acid and Artificial Sebum

The amount of adsorption of the artificial sebum was measured by the following method.

10 5.0 g of a sample was weighed out precisely in a 300 ml beaker and, as an example of fats and oils, 50.0 g of the artificial sebum were weighed out precisely. In this time, if the artificial sebum is in the semi-solid state, it is heated to have a complete solution, and then weighed out precisely. Each weighed sample was agitated vigorously for 30 minutes by a magnetic stirrer and placed stationary for 18 hours in a constant temperature chamber at 32° C. The samples were taken out from the constant temperature chamber and added to with 100 ml of petroleum ether. After the resultant product was agitated for 30 minutes, the product was filtered. This operation was repeated thrice and the resultant product was dried at 80° C. The resultant sample was weighed out precisely and was held at 500° C. for four hours and fired and the amount of adsorption was obtained from the decreased amount of the sample. Meanwhile, the amount of adsorption of oleic acid was measured by the similar manner as the method described above, without any alterations, except using oleic acid in place of the artificial sebum in the measurement of the amount of adsorption of artificial sebum described above.

B. Fatty Acid-Solidifying Capability (Solidification Starting Time)

3.6 g of oleic acid was precisely weighed out in a 50 ml beaker into which 1.0 g of the sample was charged and vigorously stirred and homogeneously mixed for ten minutes with a magnetic stirrer. The liquid mixture was set stationary and the beaker charged with the sample was tilted (inclined) and fluidized. When the beaker was returned to the original stationary set state, the time during which the deformed shape on tilting was maintained was used as the solidifying capability (solidification starting time).

C. Coefficient of Dynamic Friction (Dynamic Frictional Coefficient)

Using a friction sensory tester KES-SE manufactured by KATOTEC KK., the pressure sensitive portion and the powder surface were reciprocated three times, the coefficient of dynamic friction (the dynamic frictional coefficient) was measured.

Meanwhile, the coefficient of dynamic friction was measured before and after the adsorption of oleic acid in the powder.

D. Amount of Absorption of Oil

5.0 g of the sample was taken on a glass plate and squalane as a component similar to sebum was applied drop wise thereto. The reaction mass was kneaded homogeneously with a spatula until the sample was collected as a sole mass. At this point, as an end point, the amount of squalane dripped until this time was used as the amount of absorption of oil of the sample (ml/100 g).

(Result of Evaluation Test as to Variety of Powders)

With respect to Examples 1 to 4 and Comparative Example 1, the measured results of the amount of absorption of oil, specific surface area of powder, amount of adsorption of oleic acid, amount of adsorption of artificial sebum and fatty acid-solidifying capability (solidification starting time), and the coefficient of dynamic friction before and after the adsorption of oleic acid are shown in the following Table 2.

Moreover, amount of adsorption perm² of oleic acid and artificial sebum were obtained from values of the following Table 2 (value of specific surface area, and value of amount of adsorption of oleic acid and artificial sebum). These values are shown in the following Table 3.

TABLE 2
Result of Evaluation Test as to Variety of Powders

| | | | | | | | coefficient of | |
|---|---|---|---|---|---|---|---|---|
| | | | | amount of | amount of | | dynamic friction (×10⁻¹) | |
| | percentage of | amount of | specific | adsorption of | adsorption of | | before the | after the |
| | zinc oxide content | absorption of | surface area | oleic acid | artificial | solidification | adsorption of | adsorption of |
| samples | in powder (wt %) | oil (ml/100 g) | (m²/g) | (mg/g) | sebum (mg/g) | starting time | oleic acid | oleic acid |
| Comparative | 5 | 123 | 17.6 | 215.6 | 220.9 | 133 sec | 2.64 | 2.30 |
| Example 1 | | | | | | | | |
| Example 3 | 10 | 117 | 18.5 | 568.3 | 581.2 | 62 sec | 2.93 | 1.42 |
| Example 2 | 15 | 117 | 19.0 | 972.5 | 994.3 | 30 sec | 2.89 | 1.20 |
| Example 1 | 20 | 117 | 19.6 | 1227.0 | 1247.0 | 0 sec | 2.82 | 1.02 |
| | | | | | | (instant) | | |
| Example 4 | 30 | 113 | 19.3 | 1754.0 | 1828.0 | 35 sec | 2.92 | 1.25 |

TABLE 3
Amounts of adsorption per m² of oleic acid and artificial sebum

| | percentage of | amount of | amount of |
|---|---|---|---|
| | zinc oxide | adsorption | adsorption per |
| | content in | per m² of oleic | m² of artificial |
| samples | powder (wt %) | acid (mg/g) | sebum (mg/g) |
| Comparative | 5 | 12.2 | 12.5 |
| Example 1 | | | |
| Example 3 | 10 | 30.7 | 31.4 |
| Example 2 | 15 | 51.1 | 52.3 |
| Example 1 | 20 | 62.6 | 63.6 |
| Example 4 | 30 | 90.8 | 94.7 |

If evaluation is made from the results of the amount of absorption of oil, specific surface area, amount of adsorption of oleic acid, amount of adsorption of artificial sebum, fatty acid-solidifying capability (solidification starting time) and coefficient of dynamic friction before and after the adsorption of oleic acid, the products of the present invention (powders obtained in Examples 1 to 4) are superior to the prior product in all the evaluative items.

As compared to the amounts of adsorption of oil of 90 to 120 in the powders mixed as a filler in general, the amounts of adsorption of oil in the products of the present invention (powders obtained in Examples 1 to 4) are within the range of amounts of adsorption of oil in powders containing this filler. Therefore, the products of the present invention (powders obtained in Examples 1 to 4) can be applied as well as the general fillers.

When a comparison is made based on the amounts of adsorption of oleic acid, the amount of adsorption of oleic acid of the powder obtained in Comparative Example 1, that is, the powder in which the percentage of zinc oxide content is 5 wt %, is 215.6, whereas the amount of adsorption of oleic acid of the powder in which the percentage of zinc oxide content is 10 wt %, that is, the powder obtained in Example 3 is an approximately 2.6 times that of the powder obtained in Comparative Example 1, the amount of adsorption of oleic acid of the powder in which the percentage of zinc oxide content is 15 wt %, that is, the powder obtained in Example 2 is an approximately 4.5 times that of the powder obtained in Comparative Example 1, the amount of adsorption of oleic acid of the powder in which the percentage of zinc oxide content is 20 wt %, that is, the powder obtained in Example 1 is an approximately 5.7 times that of the powder obtained in Comparative Example 1, and the amount of adsorption of oleic acid of the powder in which the percentage of zinc oxide content is 30 wt %, that is, the powder obtained in Example 4 is an approximately 8.1 times that of the powder obtained in Comparative Example 1. Therefore, it is seen that the amounts of adsorption of the products of the present invention (powders obtained in Examples 1 to 4), that is, the powder in which the percentage of zinc oxide content is increased from 10 to 30 wt %, are extremely higher than that of the powder obtained in Comparative Example 1.

The values of the amount of adsorption of artificial sebum are approximately equivalent in the all powders to the values of the amount of adsorption of oleic acid. Therefore, when a comparison is made based on the amounts of adsorption of artificial sebum, as in the case of the comparison of the amounts of adsorption of oleic acid described above, it is seen that the amounts of adsorption of artificial sebum of the products of the present invention (powders obtained in Examples 1 to 4) are extremely higher than that of the powder obtained in Comparative Example 1.

When a comparison is made based on the solidification starting time, in the powder obtained in Comparative Example 1, it takes 2 minutes and 13 seconds (133 seconds) to solidify oleic acid, whereas, in the powder obtained in Example 3, it takes a minute and 2 seconds (62 seconds) to solidify oleic acid so oleic acid has been solidified at approximately half time. Therefore, when a content of zinc oxide in the powder is increased, it is seen that a solidification starting time is evidently shorter. Meanwhile, in the powder obtained in Example 1, the solidified oleic acid was in a state of very hard wax.

With respect to the measurement of the coefficient of dynamic friction, the measurement method thereof is the same as described above. As the equipment for measurement, a friction sensory tester KES-SE manufactured by KATOTEC KK was used. Sericite, which is powder used for a cosmetic in general, is considered to have best aesthetic feel, and the value of coefficient of dynamic friction thereof is 2.23×10⁻¹ MIU. With respect to mica, which is typical as other powder used for a cosmetic, the value of coefficient of dynamic friction thereof is 2.85×10⁻¹ MIU, and on the other hand, with respect to composite powder used for a cosmetic, the values of coefficient of dynamic friction thereof are within 2.96×10⁻¹ MIU to 3.30×10⁻¹ MIU or so. With respect to the powders of the present invention (powders obtained in Examples 1 to 4), the value of coefficient of dynamic friction thereof is within 2.8×10⁻¹ MIU to 2.9×10⁻¹ MIU or so before the adsorption of oleic acid so it is equivalent to mica used as a powder for a cosmetic in general. Therefore, it is seen that the powders of the present invention have a good aesthetic feel.

With respect to the values of the coefficient of dynamic friction of powders after the adsorption of oleic acid, the powder obtained in Comparative Example 1 thereof is 2.3×10⁻¹ MIU, whereas the powders of the present invention thereof is within 1.0×10⁻¹ MIU to 1.4×10⁻¹ MIU or so. From these results, it is seen that the powders of the prescient invention have a very smooth feeling. Therefore, it is also seen that the powders of the present invention give refreshing feeling together with smooth feeling without giving tacky feeling to skin after it is applied to skin and adsorbs sebum, even if after having been varied with time.

If evaluation is made from the results of the amount of amount of adsorption per m² of oleic acid and artificial sebum shown in the above Table 3, the amount of adsorption per m² of oleic acid of the powder obtained in Example 3 is an approximately 2.5 times that of the powder obtained in Comparative Example 1, the amount of adsorption per m² of oleic acid of the powder obtained in Example 2 is an approximately 4.1 times that of the powder obtained in Comparative Example 1, the amount of adsorption per m² of oleic acid of the powder obtained in Example 1 is an approximately 5.1 times that of the powder obtained in Comparative Example 1, and the amount of adsorption per m² of oleic acid of the powder obtained in Example 4 is an approximately 7.1 times that of the powder obtained in Comparative Example 1. Therefore, it is seen that all the amounts of adsorption per m² of oleic acid of the products of the present invention (powders obtained in Examples 1 to 4) are extremely higher than that of the powder obtained in Comparative Example 1.

### Example 6

### Relation Between Particle Size of Zinc Oxide and Solidification Starting Time

(Preperation of Mixed powder)

Mixed powder was prepared by mixing the following components (a) to (c) (sample 1):

(a) sericite (mean particle size: 8 μm, plate crystal) 190 g;

(b) hydroxyapatite obtained by the following method of preperation 37.3 g; and

(c) microporous zinc oxide (particle size measured by SEM (scanning electron microscope) observation d=0.3 μm) 12 g.

(Preparation of Hydroxyapatite)

64.8 g of calcium acetate were added to 2000 ml of purified water in the reaction vessel and the resultant mixture was heated to 85° C. When the temperature was 85° C., a solution obtained on dissolving 12 g of sodium hydroxide and 32.12 g of disodium hydrogenphosphate in 400 ml of purified water was added to the heated mixture to adjust the pH value of the solution to 9.4. A solution obtained on dissolving 2.7 g of sodium hydroxide in 270 ml of purified water was then added to the mixture to adjust the pH value thereof to 11.4. The reaction mixture was subjected to reaction and curing for one hour. When the curing was finished, the reaction mixture was cooled to 60° C. to obtain hydroxyapatite.

Next, various mixed powders were prepared by the similar manner as the method described above, without any alterations, except using zinc oxide described in the section of sample 2 or 3 in the following Table 4 in place of microporous zinc oxide (particle size measured by SEM observation d=0.3 μm) in the above sample 1 (samples 2 and 3).

TABLE 4
Zinc oxide used in the preparation of samples 1 to 3

| samples | zinc oxide |
|---|---|
| 1 | microporous zinc oxide (d*¹ = 0.3) |
| 2 | ultrafine zinc oxide (FINEX45*²) (d*¹ = 0.02) |
| 3 | ultrafine zinc oxide (FINEX75*²) (d*¹ = 0.01) |

*¹particle size measured by SEM observation (unit: μm)
*²manufactured by SAKAI KAGAKU KK

(Test Method)

With respect to the each samples obtaied by the above method of preperation, fatty acid-solidifying capability (solidification starting time) thereof were measured. Meanwhile, in this case, the solidification starting time was measured by the same manner as the measurement method of fatty acid-solidifying capability described in the above Example 5. The results are shown in the following Table 5.

TABLE 5

| samples | solidification starting time |
|---|---|
| 1 | not less than 2 hours |
| 2 | not less than 2 hours |
| 3 | 60 minutes and 8 seconds |

As shown in the above Table 5, in case that the particle size of zinc oxide was large, oleic acid did not solidify even after passage of not less than 2 hours. Meanwhile, it was decided that the measurement of time of solidification (solidification starting time) was conducted until 2 hours. From this result (Table 5), it is seen that the solidification starting time is affected by the particle size of zinc oxide. That is, in the mixed powder, in case that the particle size of zinc oxide is not less than 0.02 μm, it is pressumed that not less than 2 hours of time of solidification is required, while, in case that the particle size (mean particle size) of zinc oxide is 0.01 μm, the fatty acid (oleic acid) is solidified at approximately half of the time.

From the above, it is considered that the particle size of zinc oxide is one of factors exerting an effect on a speed of the solidification (solidification speed).

### Example 7

### Comparison of Solidification Starting Time of Powders of the Present Invention and Comparative Product to that of Mixed Powders (the Following Contrastive Examples 1 to 5)

### Preparation of Powders (Contrastive Examples 1 to 5

According to the method of preparation described in the above Example 6, contrastive products (mixed powders; Contrastive Examples 1 to 5) were prepared. Then, each of powders was prepared by changing each component and the usage thereof into those of the following Table 6. On the other hand, the preparation of hydroxyapatite was also conducted by changing each component and the usage thereof into those of the following Table 7. As a zinc oxide, ultrafine zinc oxide (FINEX 75) (particle size mesured by SEM observation d=0.01 μm) was used. Meanwhile, in the percentage of zinc oxide content, Example land Contrastive Example 1, Example 2 and Contrastive Example 2, Example 3 and Contrastive Example 3, Example 4 and Contrastive Example 4, and Comparative Example 1 and Contrastive 5 Example 5 are the same respectively.

TABLE 6
Usage of each components in production of various mixed powders

| | Contrastive | Contrastive | Contrastive | Contrastive | Contrastive |
|---|---|---|---|---|---|
| | Example 1 | Example 2 | Example 3 | Example 4 | Example 5 |
| | (containing | (containing | (containing | (containing | (containing |
| | 20%* of | 15%* of | 10%* of | 30%* of | 5%* of |
| components | zinc oxide) | zinc oxide) | zinc oxide) | zinc oxide) | zinc oxide) |
| sericite (mean particle size: | 152 | 171 | 190 | 121.6 | 190 |
| 8 μm, plate crystal) (g) | | | | | |
| hydroxyapatite (g) | 29.8 | 33.7 | 37.4 | 23.9 | 37.3 |
| ultrafine zinc oxide (FINEX75) | 45.4 | 36.1 | 25.3 | 62.4 | 12.0 |
| (particle size measured by SEM | | | | | |
| observation d = 0.01 μm) (g) | | | | | |

*wt %

TABLE 7
Usage of each components in preparation of hydroxyapatite

| | | Contrastive | Contrastive | Contrastive | Contrastive | Contrastive |
|---|---|---|---|---|---|---|
| | | Example 1 | Example 2 | Example 3 | Example 4 | Example 5 |
| | | (containing | (containing | (containing | (containing | (containing |
| | | 20%* of | 15%* of | 10%* of | 30%* of | 5%* of |
| components | | zinc oxide) | zinc oxide) | zinc oxide) | zinc oxide) | zinc oxide) |
| purified water (ml) | | 2000 | 2000 | 2000 | 2000 | 2000 |
| calcium acetate (g) | | 58.32 | 64.8 | 41.5 | 64.8 | 51.84 |
| sodium hydroxide- | purified water (ml) | 320 | 360 | 400 | 284.2 | 400 |
| disodium | sodium hydroxide (g) | 9.6 | 10.8 | 12 | 8.5 | 12 |
| hydrogenphosphate | disodium | 25.7 | 28.9 | 32.12 | 22.8 | 32.12 |
| aqueous solution | hydrogenphosphate (g) | | | | | |
| sodium hydroxide | purified water (ml) | 226 | 243 | 270 | 187.6 | 270 |
| aqueous solution | sodium hydroxide (g) | 2.26 | 2.43 | 2.7 | 1.89 | 2.7 |

*wt %

(Test Method)

With respect to the powders obtaied in the above Contrastive Examples 1 to5, the fatty acid-solidifying capability (solidification starting time) thereof were measured. Meanwhile, in this case, the solidification starting time was measured by the similar manner as the measurement method of fatty acid-solidifying capability described in the above Example5. The results are shown in the following Table 8.

TABLE 8
Solidification starting times of, Examples 1 to 4 and
Comparative Example and Contrastive Examples 1 to 5

| | composite powder | | | | |
|---|---|---|---|---|---|
| | | | | | Comparative |
| | Example 1 | Example 2 | Example 3 | Example 4 | Example 1 |
| percentage of | 20 | 15 | 10 | 30 | 5 |
| zinc oxide | | | | | |
| content in | | | | | |
| powder (wt %) | | | | | |
| solidification | 0 sec | 30 sec | 1 min and | 35 sec | 2 min and |
| starting time | (instant) | | 2 sec | | 13 sec |

| | mixed powder | | | | |
|---|---|---|---|---|---|
| | Contrastive | Contrastive | Contrastive | Contrastive | Contrastive |
| | Example 1 | Example 2 | Example 3 | Example 4 | Example 5 |
| percentage of | 20 | 15 | 10 | 30 | 5 |
| zinc oxide | | | | | |
| content in | | | | | |
| powder (wt %) | | | | | |
| solidification | 13 min and | not | 25 min and | 5 min and | 60 min and |
| starting time | 24 sec | measured | 48 sec | 54 sec | 8 sec |

As shown in the above Table 5, the solidification starting times of the powders obtained in Examples 1 to 4 and Comparative Example 1 (composite powders) are shorter than those of the powders obtained in Contrastive Examples 1 to 5 (mixed powders). In particular, as described above, the solidification starting times of the powders of the present invention (obtained in Examples 1 to 4) are shorter as compared to that of Comparative Example 1. Therefore, it is seen that the constitution, in which a layer of hydroxyapatite exists directly on the surface of a base substance and a layer of zinc oxide exists directly on the surface of said layer of hydroxyapatite, is superior to that, in which the same composition as the powder of the present invention is simply mixed, in a solidifying capability. In particular, it is seen that the composite powders are extremely superior in a solidifying capability. It is presumed that this is because, since hydroxyapatite is congealed on sericite (base substance) as a primary particle by a spacer effect of the sericite (base substance), and further, ultrafine zinc oxide is congealed on the hydroxyapatite as a primary particle by a spacer effect of the hydroxyapatite, in the three-layer constitution of the composite powder, unsaturated fatty acid or sebum, which is adsorbed to this hydroxyapatite in a state of un-solidification, forms a primary particle by the spacer effect of the hydroxyapatite, and the solidification proceeds smoothly together actions of adsorbing and solidifying the ultrafine zinc oxide, which is adjacent to this hydroxyapatite, and as a result, a speed of the solidification is fast. On the other hand, in case of the mixed powder, hydroxyapatite prepared by the preparation of hydroxyapatite described above or ultrafine zinc oxide purchased from a market flocculates, and these are in a state of scatteredness in sericite. That is, both hydroxyapatite, which adsorbs unsaturated fatty acid or sebum and flocculates in a state of un-solidification, and ultrafine zinc oxide, which adsorbs unsaturated fatty acid or sebum and flocculates in a state of solidification, are in a state of scatteredness in sericite. It is considered that since the actions of adsorbing and solidifying the unsaturated fatty acid or sebum do not proceed smoothly between such hydroxyapatite flocculated and ultrafine zinc oxide flocculated, a speed of the solidification is slow. That is, it is presumed that since effects of sericite, which are exerted in a composite powder produced by liquid phase action and are an effect of fixing hydroxyapatite as a primary particle and enhancing property and function of the particle, are not exerted in the mixed powder but a speed of the solidification becomes slow by inhibition of the process between hydroxyapatite and ultrafine zinc oxide (the actions of adsorbing and solidifying the unsaturated fatty acid or sebum).

### Example 8

### Preparation of Powder Foundation

A powder foundation was prepared according to the following method for preparation, based on the composition of the following Table 9.

TABLE 9
Composition of Powder foundation (unit: parts by weight)

| components | | amount |
|---|---|---|
| powder | powder obtained in Example 1* | 13.5 |
| components | boron nitride | 3.0 |
| | sericite treated with silicone | 15.3 |
| | sericite treated with lecithin | 7.0 |
| | sericite treated with amino acid | 5.0 |
| | sericite treated with fluorine | 11.0 |
| | cross polymer of | 7.0 |
| | (dimethicone/vinylmethicone/methicone) | |
| | nylon powder | 7.0 |
| | titanium dioxide treated with silicone | 16.0 |
| | red iron oxide treated with silicone | 0.4 |
| | yellow iron oxide treated with silicone | 2.2 |
| | iron black treated with silicone | 0.4 |
| | methyl paraben | 0.2 |
| oily | squalane | 1.20 |
| components | solid paraffin | 1.04 |
| | petrolatum (vaseline) | 2.00 |
| | stearic acid | 0.64 |
| | dimethyl polysiloxane | 5.48 |
| | glyceride tri-2-ethyl hexanoate | 1.60 |
| | d-δ-tocopherol | 0.04 |

*To the powder obtained in Example 1, a solution of methylhydrogen and isopropylalcohol was added for moistening this powder homogeneously, and subsequently, the mixture thus obtained was cooled after heating. The mixture thus obtained was used as a powder.

(Method for Preparing Powder Foundation)

The above powder components were mixed in a Henschel mixer and taken out. Subsequently, the mixture so taken out was transferred to a pulverizer and pulverized therein. This pulverized mixture was charged into a Henschel mixer and added to with oily agents, which were mixed through heating in advance, for coating. The mixture obtained was powdered by using a pulverizer. After this pulverized mixture thus obtained was charged into a metal mold, it was pressure molded to obtain an objective powder foundation.

### Example 9

### Preparation of Lotion

A lotion was prepared according to the following method for preparation, based on the composition of the following Table 10.

TABLE 10
Composition of Lotion (unit: parts by weight)

| components | | amount |
|---|---|---|
| phase A | glycerine | 5 |
| | 1,3-butylene glycol | 5 |
| | carboxy vinyl polymer | 0.5 |
| | potassium hydroxide | microdose |
| | coleus extract | 0.3 |
| | decaglyceryl monoisostearate | 1 |
| | decaglyceryl triisostearate | 0.5 |
| | ethanol | 1 |
| | POE (40) hardened castor oil | 0.5 |
| | seaweed extract powder | 0.2 |
| | trimethyl glycine | 3 |
| phase B | bentonite | 0.2 |
| | powder obtained in Example 3* | 1.0 |
| | perfume | 0.2 |
| | purified water | to 100 |

(Method for Preparing Lotion)

Bentonite was dispersed in purified water. Subsequently, into the dispersion thus obtained, powder obtained in Example 3 was dispersed, and further perfume was added thereto for preparing phase B. To this phase B obtained, the mixture of the above components of phase A, which was mixed in advance, was added. After this mixture thus obtained was mixed and stirred, it was charged into a package to obtain an objective lotion.

### Example 10

### Preparation of Lipstick

A lipstick was prepared according to the following method for preparation, based on the composition of the following Table 11.

TABLE 11
Composition of Lipstick (unit: parts by weight)

| components | | amount |
|---|---|---|
| component A | candelilla wax | 2 |
| | paraffin wax | 6 |
| | ceresin | 1 |
| | powder obtained in Example 2* | 10 |
| | petrolatum | 3 |
| | purified lanolin | 10 |
| | diisostearyl stearate malate | 20 |
| | neopentyl glycol dicaprate | 30 |
| | squalane | 10 |
| component B | Red-202 | 4 |
| | Yellow 4 Al lake | 2 |
| | titanium dioxide | 2 |

*The powder obtained in Example 2 was dispersed into aqueous phase, in which water-soluble amino acid salt was dissolved, and subsequently, water-soluble inorganic salt was dropped thereto. Subsequently, the mixture thus obtained was dried after filtering. The mixture thus obtained (substance treated with amino acid) was used as a powder.

(Method for Preparing Lipstick)

The above raw materials in component A were homogeneously mixed through melt by heating. Subsequently, to the mixture thus obtained, the above color raw materials (component B), which was mixed in advance, were added. The mixture thus obtained was homogeneously dispersed in a state of heating by disperser. Subsequently, after the dispersion thus obtained was degassed, the dispersion obtained was poured into a mold to obtain an objective lipstick.

### Example 11

### Preparation of Face Powder

A face powder was prepared according to the following method for preparation, based on the composition of the following Table 12.

TABLE 12
Composition of Face powder (unit: parts by weight)

| components | | amount |
|---|---|---|
| component A | zinc myristate | 3 |
| | talc treated with calcium distearate | 14 |
| | burned mica treated with silicone | 13 |
| | powder obtained in Example 4* | 40 |
| | spherical methylsiloxane net polymer powder | 3 |
| | mica titaninum coated with aluminium oxide | 2 |
| | organo polysiloxane powder | 15 |
| | iron oxide | 2 |
| | boron nitride | 1 |
| | N-acylated lysine | 2 |
| component B | glyceryl triisooctanoate | 2 |
| | liquid paraffin | 1 |
| | squalane | 2 |

*The powder obtained in Example 4 was added to perfluoro alkyl silane, which was dissolved in methyl ethyl ketone, and thereafter the mixture obtained was homogeneously stirred. Subsequently, the mixture thus obtained was heat-treated and thereafter cooled. The mixture thus obtained (substance treated with fluorine) was used as a powder.

(Method for Preparing Face Powder)

The components of the above component A were mixed in naughter mixer and taken out. Subsequently, the mixture so taken out was transferred to a pulverizer and pulverized therein. This pulverized mixture was charged into a Henschel mixer and added to with the components of the above component B, which were mixed in advance. The mixture was stirred, and thereafter powdered by using a pulverizer. This pulverized mixture thus obtained was charged into a package to obtain an objective face powder.

### Example 12

### Preparation of Emulsion

An emulsion was prepared according to the following method for preparation, based on the composition of the following Table 5 13.

TABLE 13
Composition of Emulsion (unit: parts by weight)

| components | | amount |
|---|---|---|
| phase A | decamethylcyclopentasiloxane | 16.5 |
| | stearic acid | 1.2 |
| | propyl paraben | 0.2 |
| | δ-tocopherol | 0.02 |
| | fatty acid saccharide | 1.2 |
| | microcrystalline wax | 0.25 |
| | sorbitan distearate | 0.8 |
| | polyglycerine polyoxybutylene stearyl ether | 0.3 |
| | glycerine monoisostearate | 0.5 |
| | glycerine monostearate | 1.5 |
| | polyethylene glycol monostearate | 0.35 |
| | sorbitan sesquioleate | 0.4 |
| | powder obtained in Example 1* | 2.5 |
| | talc | 10 |
| | titanium dioxide | 5.0 |
| phase B | 1,3-butylene glycol | 2.0 |
| | glycerine | 4.0 |
| | ethyl paraben | 0.2 |
| | xanthane gum | 0.05 |
| | L-arginine | 0.4 |
| | purified water | 52.63 |

*To the powder obtained in Example 1, the dissolved substance, which was obtained by dissolving lecithin with hot water, was added. Subsequently, the mixture thus obtained was homogeneously mixed. The mixture thus obtained (substance treated with lecithin) was used as a powder.

(Method for Preparing Emulsion)

The components of the above phase A were dissolved at 80° C. and homogeneously dispersed. Next, a mixture of components of phase B, which was homogeneously mixed, was heated to 80° C. and added to the mixture of phase A and emulsified through stirring. The mixture obtained was cooled to 40° C., and charged into a package to obtain an objective emulsion.

### Example 13

### Preparation of Hair-Growth Medicine

A hair-growth medicine was prepared according to the following method for preparation, based on the composition of the following Table 14.

TABLE 14
Composition of Hair-growth medicine (unit: parts by weight)

| components | | amount |
|---|---|---|
| phase A | purified water | 38.0 |
| | pyridoxine hydrochloride | 0.05 |
| | citric acid | 0.15 |
| | sodium citrate | 0.10 |
| | 1,3-butylene glycol | 2.0 |
| | powder obtained in Example 1 | 0.2 |
| phase B | phenylethyl alcohol denatured alcohol | 52.2 |
| | acetyl-dl-α-tocopherol | 0.1 |
| | menthol | 0.1 |
| | diphenhydramine chloride | 0.1 |
| | diethoxyethyladipate | 3.0 |
| | polyoxyethylene hardened castor oil | 1.0 |
| | purified water | 3.0 |

(Method for Preparing Hair-Growth Medicine)

To the above components of phase A, which were dissolved and dispersed in advance, the above components of phase B, which were dissolved in advance, were added. The mixture obtained was homogeneously mixed through stirring. Subsequently, the mixture thus obtained was charged into a package to obtain an objective hair-growth medicine.

### Example 14

### Preparation of Body Deodorant

A body deodorant was prepared according to the following method for preparation, based on the composition of the following Table 15.

TABLE 15
Composition of the Body Deodorant (unit: parts by weight)

| components | | |
|---|---|---|
| No. | components | amount |
| 1 | talc treated with silicone | 38.0 |
| 2 | inventive substance (Example 1) | 40.0 |
| 3 | cyclic dimethyl polysiloxane | 20.0 |
| 4 | cetyl octanoate | 1.0 |
| 5 | glycerine tri-2-ethyl hexanoate | 1.0 |

(Method for Preparing Body Deodorant)

The components 1 and 2 were mixed in a Henschel mixer and powdered by a pulverizer. The pulverized mass was transferred to a Henschel mixer and added to with a liquid mixture of the components 3 to 5 as the oily components for mixing. The resultant mixture then was powdered by a pulverizer to prepare a body deodorant.

### Example 15

### Amount of Adsorption of Oleic Acid and Amount of Adsorption of Artificial Sebum

In the powder foundation obtained in aforementioned Example 8, the amount of adsorption of oleic acid and the amount of adsorption of artificial sebum were measured and compared to conventional (prior) products. Meanwhile, in this case, they were measured by the same way as the mesurement method of the amount of adsorption of oleic acid and artificial sebum described in Example 5. The results are shown in Table 16.

TABLE 16
Amount of adsorption of oleic acid and amount of
adsorption of artificial sebum in the inventive
substance and variety of Conventional products

| | amount of | amount of |
|---|---|---|
| | adsorption of | adsorption of |
| | oleic acid | artificial |
| samples | (mg/g) | sebum (mg/g) |
| inventive substance | 451.2 | 356.9 |
| (Example 8) | | |
| Conventional product (A) | 77.2 | 54.5 |
| Conventional product (B) | 289.7 | 70.4 |
| Conventional product (C) | 66.9 | 63.9 |
| Conventional product (D) | 86.6 | 96.6 |

The inventive substance (Example 8) only exhibited high values of the amounts of adsorption of both oleic acid and the artificial sebum. The conventional (prior) product (B) and the inventive substance exhibited specifically an adsorption for oleic acid (free fatty acid). In particular, it may be seen that the inventive substance exhibited an apparently high adsorbent effect of oleic acid as compared to the conventional product (B), of which the amount of adsorption was the highest in the above conventional products. Similarly, it may also be seen that the inventive substance exhibited adsorbent effects for artificial sebum, which are apparently superior to those of the conventional products.

### Example 16

### Evaluation of Usability

The powder foundation obtained in Example 8 was evaluated. As a comparative substance (Comparative Example 2), a powder foundation prepared in accordance with the similar manner as the method in Example 8 except substituting a sericite treated with silicone for all of the powder of Example 1 was used. For evaluation method, six expert peoples in a panel actually used the powder foundation and, after use, five-stage evaluation was conducted as to the following evaluation items in accordance with the following standard:

(Evaluation Standard)

| | 1 | 2 | 3 | 4 | 5 |
|---|---|---|---|---|---|
| long wear | bad | | [symbol] | | good |
| transparency | no | | [symbol] | | yes |
| extension on skin | bad | | [symbol] | | good |
| makeup finish | not | | [symbol] | | beautiful |
| | beautiful | | | | |
| affixture | no | | [symbol] | | yes |
| adhesion | bad | | [symbol] | | good |
| smooth feeling after | no | | [symbol] | | yes |
| passage of 5 hours | | | | | |

(Result of Evaluation)

| | | Comparative |
|---|---|---|
| items of evaluation | Example 8 | Example 2 |
| long wear | 4.9 | 1.0 |
| transparency | 4.2 | 4.2 |
| extension on skin | 3.6 | 3.8 |
| makeup finish | 4.8 | 2.5 |
| affixture | 4.6 | 2.4 |
| adhesion | 4.5 | 2.1 |
| smooth feeling after | 5.0 | 1.0 |
| passage of 5 hours | | |

From the results of the above, it may be seen that the powder foundation of the present invention is extremely superior in effects of cosmetic, such as long wear, makeup finish and the like.

Effect of Invention

The present invention can provide a powder which is more sperior in properties for cosmetic (effects of cosmetic), such as improvement of long wear for makeup, suppression of “shiny” look and improvement of aesthetic feel, and adsorbent property and solidifying capability of free fatty acids, in particular unsaturated fatty acids, and sebum. This powder may be conveniently used for cosmetics. The present invention also provides a cosmetic and a sebum-adsorbent having these superior effects by the use of these powders. Moreover, the powder in the present invention exhibits the operation of adsorbing, solidifying, or congealing the body odor components and hence the present invention also provides a body deodorant employing the inventive powder. Therefore, the present invention is industrially extremely useful especially in the field of cosmetics.

It should be noted that other objects, features and aspects of the present invention will become apparent in the entire disclosure and that modifications may be done without departing the gist and scope of the present invention as disclosed herein and claimed as appended herewith.

Also it should be noted that any combination of the disclosed and/or claimed elements, matters and/or items might fall under the modifications aforementioned.

What is claimed is:

1. A powder having a base substance usable as a powder for cosmetics, a layer of hydroxyapatite which exists directly on the surface of said base substance and a layer of zinc oxide which exists directly on the surface of said layer of hydroxyapatite, and containing 5 to 30% by weight of the hydroxyapatite and 10 to 50% by weight of the zinc oxide based on the total weight of said powder.

2. The powder as defined in claim 1, which is used for cosmetics.

3. The powder as defined in claim 1, which has an adsorbent property for sebum components.

4. The powder as defined in claim 1, wherein said base substance contains an inorganic powder and contains any one of a clay mineral, a metal hydroxide, a metal oxide, a composite material thereof, and composite material (s) of one or more of these inorganic powders with an organic powder, wherein said clay mineral may be a synthetic material.

5. The powder as defined in claim 1, wherein said zinc oxide is at least one of low crystalline zinc oxide and amorphous zinc oxide.

6. The powder as defined in claim 1 or 5, wherein said zinc oxide has a crystalline size of 1000 Å at the longest as obtained from Scherrer's equation.

7. The powder as defined in claim 1, wherein said base substance has a lamellar shape, a scale-like shape, a plate-like shape or a bar-like shape.

8. The powder as defined in claim 1, wherein the thickness of said layer of hydroxyapatite is 0.05 to 10 μm.

9. The powder as defined in claim 1, the particle of said hydroxyapatite is comprised of spicular particle, and the length thereof is 0.1 to 10 μm and the breadth thereof is 0.01 to 0.06 μm.

10. The powder as defined in claim 1, 8 or 9, wherein the mean particle size of said base substance is 0.1 to 600 μm and the mean particle size of said zinc oxide is 0.001 to 1 μm.

11. The powder as defined in claim 1, wherein the mean particle size of said base substance is 0.1 to 600 μm, the mean particle size of said zinc oxide is 0.01 to 0.03 μm and the particle of said hydroxyapatite is comprised of spicular particle, and the length thereof is 0.1 to 2 μm and the breadth thereof is 0.02 to 0.04 μm.

12. The powder as defined in claim 1, or 11, the specific surface area of said zinc oxide is 105 to 500 m² /g.

13. A cosmetic comprising the powder as defined in any one of claims 1 to 3.

14. The cosmetic as defined in claim 13, containing 0.01 to 90 wt % of said powder.

15. A sebum-adsorbent agent comprising the powder as defined in claim 1 or 3.

16. A body deodorant comprising the powder as defined in claim 1 or 3.
